(19) United States
(12) Patent Application Publication
(10) Pub. No.: US 2006/0149137 A1
(43) Pub. Date: Jul. 6, 2006

(54) Easily placeable and removable wound retractor

(75) Inventors: Edward D. Pingleton, San Juan Capistrano, CA (US); Jeremy J. Albrecht, Ladera Ranch, CA (US)

Correspondence Address: APPLIED MEDICAL RESOUCES CORPORATION, 22872 Avenida Empresa, Rancho Santa Margarita, CA, 92688, US

(73) Assignee: Applied Medical Resources Corporation

(21) Appl. No.: 11/029,606

(22) Filed: Jan. 5, 2005

(51) Int. Cl. A61B 1/32

(52) U.S. Cl. 600/208

(57) ABSTRACT

The invention is directed to a surgical wound retractor for retracting and sealing an incision and forming a functional opening or channel through which a surgical procedure may be executed. The wound retractor provides a path for a surgeon to insert his hand and/or instruments through the opening formed by the wound retractor. The wound retractor is sized and configured to be easily placed through a small incision and removed without further insult to the body tissue adjacent to the incision. The wound retractor is adapted to dilate a surgical wound incision to a desired diameter, and comprises a first ring having a diameter greater than the desired diameter of the wound incision and being adapted for disposition interiorly of the wound incision; a second ring having an annular axis and a diameter greater than the desired diameter of the wound incision and being adapted for disposition exteriorly of the wound incision; and a flexible sleeve disposed in a generally cylindrical form between the first ring and the second ring, the first ring having at least one notch to facilitate folding or collapsing of the first ring during insertion and removal of the first ring through the wound incision. The first ring may further comprise a second notch disposed on an opposing end of the first notch to further facilitate folding or collapsing of the first ring. With this aspect, the first ring is folded by squeezing between the first and second notches during insertion and removal of the retractor from the incision. In another aspect, the wound retractor may further comprise a tether having a length, a first end attached to the first ring interiorly of the sheath, and a second end disposed outside of the wound incision, wherein the tether facilitates removal of the first ring by pulling on the second end to retrieve the first ring through the wound incision.

### BACKGROUND OF THE INVENTION

1. Field of the Invention

This invention generally relates to medical devices and, more specifically, to a surgical wound retractor that is easily placeable and removable from a wound incision.

2. Discussion of the Prior Art

Surgical procedures generally require that an incision be made through a body wall to provide access into a body cavity such as an abdominal cavity. A small incision is made in most cases and, consequently, it is often necessary to retract the small incision to maintain an adequate working area. In addition, it is important to seal the insulted regions of body tissue against the working space or channel through which instruments and contaminated tissue may pass.

Generally such retractors are made of metal or plastic rings that may have extensions that reach through a wound and subsequently expand to retract the actual wound. In recent years, flexible wound retractors have been introduced that greatly simplify the retraction of surgical wound sites. Generally these retractors comprise an outer flexible or non-flexible ring connected to a flexible inner ring by a cylindrical sheath or membrane. The inner ring is inserted through a wound and subsequently into a body cavity where it is allowed to assume its original shape, usually oval or circular. Tension is then placed on the cylindrical sheath to retract the wound site; the sheath may be shortened in length about the outer ring.

While effective in retracting surgical wound sites, the current wound retractors are generally limited for larger wound sites due to their relatively wide and rigid proportions. In particular, an incision has to be large enough to insert the inner ring of the wound retractor. For instance, an incision of five centimeters or larger could be retracted using an outer ring of about 6 inches in diameter having a cord diameter of about 0.25″ and an inner ring of about the same size. The two rings are connected by a gas-tight sheath having a wall thickness of about 0.003″ to about 0.010″. These proportions allow appropriate insertion and removal of the wound retractor from the incision or wound site. However, if the wound retractor or the incision were to be scaled down in size from the example above because of surgical needs, there is great difficulty in placement and removal of the retractor. Accordingly, there is a need for a small-sized surgical wound retractor that is easily placed and easily removed from a small wound incision or site.

### SUMMARY OF THE INVENTION

The invention is directed to a surgical wound retractor for retracting and sealing an incision and forming a functional opening or channel through which a surgical procedure may be executed. The wound retractor provides a path for a surgeon to insert his hand and/or instruments through the opening formed by the wound retractor. The wound retractor according to the invention is sized and configured to be easily placed through a small incision and removed without further insult to the body tissue adjacent to the incision.

In one embodiment, the wound retractor is being adapted to dilate a surgical wound incision to a desired diameter, the wound retractor comprising a first ring having a diameter greater than the desired diameter of the wound incision and being adapted for disposition interiorly of the wound incision; a second ring having an annular axis and a diameter greater than the desired diameter of the wound incision and being adapted for disposition exteriorly of the wound incision; and a flexible sleeve disposed in a generally cylindrical form between the first ring and the second ring, the first ring having at least one notch to facilitate folding or collapsing of the first ring during insertion and removal of the first ring through the wound incision. The first ring may further comprise a second notch disposed on an opposing end of the first notch to further facilitate folding or collapsing of the first ring. With this aspect, the first ring is folded by squeezing between the first and second notches during insertion and removal of the retractor from the incision. In another aspect, the wound retractor may further comprise a tether having a length, a first end attached to the first ring interiorly of the sheath, and a second end disposed outside of the wound incision such that the tether facilitates removal of the first ring through the wound incision. With this aspect, the first end of the tether may be attached at the notch. It is appreciated that as the tether is pulled through the wound incision, the first ring deforms to a low-profile.

In another aspect, the first ring may have an annular cross-section that is circular, oval, D-shape, or of any geometrical shape with multiple sides. With the present aspect of the invention, the second ring may be rolled over itself and around the annular axis to provide the sleeve with a radial retraction force sufficient to stretch the incision to the desired diameter. That is, any portion of the sleeve extending outside the incision can be easily rolled onto the second ring to tightly seal the sides of the surgical wound. The second ring may include surfaces that are easy to grip and turn to allow the user to manually turn the second ring and roll up the sleeve with ease. The sleeve may be made of a material that is flexible and impermeable to fluids and bacteria.

In another aspect, a surgical access device is disclosed comprising a wound retractor including a first ring having a diameter greater than the desired diameter of the wound incision and being adapted for disposition interiorly of the wound incision, a second ring having an annular axis and a diameter greater than the desired diameter of the wound incision and being adapted for disposition exteriorly of the wound incision, and a flexible sleeve disposed in a generally cylindrical form between the first ring and the second ring; and an obturator having at least one of an insertion portion and a removal portion to facilitate insertion and removal of the first ring through the wound incision. The access device may further comprise a cannula to further facilitate insertion of the first ring through the wound incision. With this aspect, the first ring is folded or collapsed into the cannula prior to insertion through the wound incision. The obturator can be formed of a rigid material including a plastic or metallic material. The obturator may further comprise a mechanical means having a sliding member that closes off at least one of the insertion portion and the removal portion.

In another embodiment of the invention, there is disclosed a surgical access device facilitating a sealing relationship with an instrument extending through the device and into an incision, the access device comprising a first ring being adapted for disposition interiorly of the incision; a second ring having an annular axis and being adapted for disposition exteriorly of the incision; a flexible sleeve connecting the first ring and the second ring; and a valve structure disposed relative to the incision to securely receive the second ring, the first ring having at least one notch to facilitate folding or collapsing of the first ring during insertion and removal of the first ring through the incision. With this aspect of the invention, the valve structure comprises a gel cap and an abdominal base. The gel cap may further comprise a gel pad and a circumferential cap ring, and the abdominal base may further comprise a rounded end surface along its inner diameter to secure the second ring.

Another embodiment of the invention is a method for operating the surgical wound retractor, the method comprising the steps of providing a flexible sleeve having a first end attached to a first ring and a second end attached to a second ring, the first ring having at least one notch to facilitate folding or collapsing of the first ring; folding the first ring at the notch and inserting the first ring and the sleeve into a patient's body cavity so as to provide a channel from the outside to the inside of the patient's body; drawing the second end of the sleeve upwards and radially outward or inward thereby drawing the first ring tightly against the inner surface of the body cavity, and thereby retracting the adjacent tissue and producing a tightly sealed opening in the body cavity during surgery; and after surgery, grabbing the first ring so as to fold it at the notch and pulling the first ring and the sleeve through the wound opening. With this aspect, the second ring may have properties to roll over itself and around an annular axis to shorten the sleeve in predetermined increments, and the method may further comprise the step of rolling the sleeve by gripping the second ring and turning it around the annular axis in either an outward or inward direction until the sleeve abuts the outer edge of the wound incision. In another aspect, the invention may further comprise a tether having a length, a first end attached to the first ring interiorly of the sheath, and a second end disposed outside of the wound incision, wherein the tether facilitates removal of the first ring through the wound incision, and the method may further comprise the step of pulling the tether at the second end to remove the first ring from the wound incision.

These and other features and advantages of the invention will become more apparent with a discussion of embodiments in reference to the associated drawings.

### DESCRIPTION OF THE DRAWINGS

FIG. 1 is a side view of a surgical procedure illustrating the placement of a wound retractor of the invention;

FIG. 2 is a side view of a surgical procedure illustrating the removal of the wound retractor of the invention;

FIG. 3 is a perspective view of the wound retractor of the invention;

FIG. 4 is a top view of an embodiment of the inner flexible ring in an open condition;

FIG. 5 is a top view of an embodiment of the inner flexible ring in a folded condition for insertion and removal into a wound site;

FIG. 6 is a top view of another embodiment of the inner flexible ring in an open condition;

FIG. 7 is a top view of another embodiment of the inner flexible ring in a folded condition for insertion and removal into a wound site;

FIG. 8 illustrates another embodiment of a wound retractor of the invention having a tether configured to aid in the removal of the inner ring from a body cavity;

FIG. 9 illustrates the wound retractor of FIG. 8 having a tether configured to aid in the removal of the inner ring from a body cavity;

FIG. 10 is a cross-section view of the cord of the flexible inner ring having a “D” shape;

FIG. 11 illustrates a perspective view of the wound retractor adapted to be used with a hand assisted laparoscopic (HAL) device in accordance with the invention;

FIG. 12(a) is a side view of a surgical procedure illustrating the placement of the inner flexible ring using an obturator in accordance with another aspect of the invention;

FIG. 12(b) is a side view of a surgical procedure illustrating the placement of the inner flexible ring through a cannula using the obturator of the invention;

FIG. 12(c) is a side view of a surgical procedure illustrating the removal of the inner flexible ring using the obturator of the invention;

FIG. 12(d) is a side view of an obturator in accordance with another aspect of the invention having a recessed diameter to facilitate insertion and removal of the inner flexible ring; and

FIG. 12(e) is a side view of an obturator in accordance with another aspect of the invention having an insertion hook and a removal hook to facilitate insertion and removal of the inner flexible ring.

### DESCRIPTION OF THE INVENTION

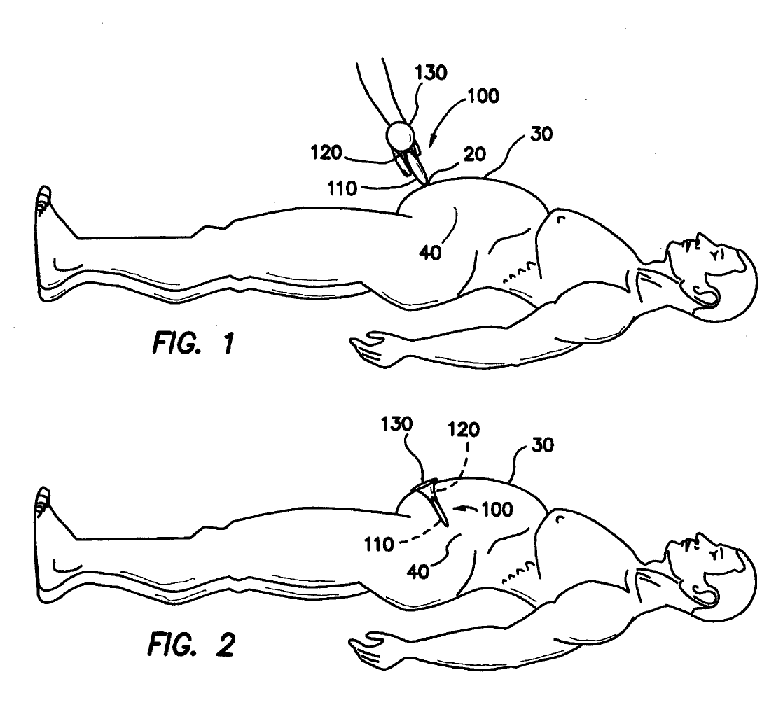

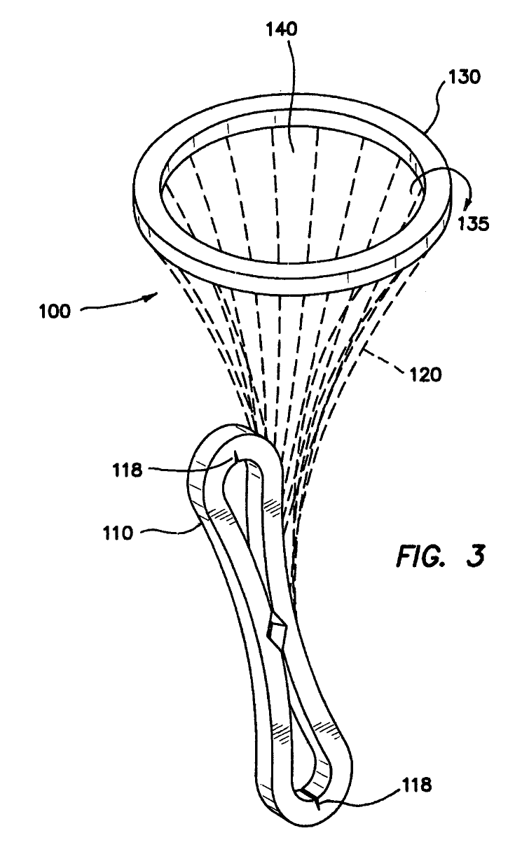

Referring to FIGS. 1 and 2, there are shown a minimally invasive surgical procedure in which a small surgical wound retractor 100 is used. As illustrated in FIG. 3, the wound retractor 100 comprises an outer ring 130, an inner flexible ring 110, and a distensible sheath or sleeve 120 connecting the outer ring 130 and the inner flexible ring 110. The sheath 120 may be attached to the outer ring 130 and the inner flexible ring 110 by heat seal, adhesive or mesh fixation. The outer ring 130 and the inner ring 110 are preferably made of a material of sufficient hardness to retain their shape after twisting and rolling of the rings. That is, the material must be compliant enough to allow the outer ring 130 to be turned around its annular axis as further discussed in applicant's patent application Ser. No. (to be assigned), filed on Nov. 30, 2004, claiming priority to international application PCT/US03/17389, filed on Jun. 3, 2003, both entitled “Wound Retractor,” both of which are incorporated herein by reference. The shape of the outer ring 130 affects both its ability to grip and to provide stability during and after adjustment. The sheath 120 is preferably made of a material that is flexible and impermeable to fluids and bacteria.

Referring back to FIGS. 1 and 2, the inner flexible ring 110 of the retractor 100 is placed through an incision 20 in a body wall 30 and subsequently into a body cavity 40. The inner ring 110 is then allowed to assume its normal shape, typically oval or circular but can be of any geometrical shape, inside the body cavity 40 and is then drawn against an interior wall surface so that the sheath 120 is formed through the incision 20. The sheath 120 is then tensioned between the inner ring 110 and outer ring 130 of the retractor 100. This will provide wound retraction and wound sealing between the incised tissue and a “working channel” 140 (see FIG. 3) of the retractor 100.

In accordance with the invention, the wound retractor 100 is axially adjustable in increments. In particular, the upper end of the sheath 120 can be wrapped around the outer ring 130 so as to tightly seal the sides or edges of the incision. The shape of the outer ring 130 allows it to roll about itself. The outer ring 130 also provides for incremental shortening of the sheath 120 and for stability after installation. Specifically, since the upper end of the sheath 120 is connected to the outer ring 130, the sheath 120 can be drawn upwards and radially outward or inward, thereby drawing the inner ring 110 tightly against the interior wall surface of the abdominal wall. Moreover, the intermediate portion of the sheath 120 is drawn tightly against the sides and edges of the wound opening, thereby retracting the adjacent tissue and producing a tightly sealed opening in the body cavity 40. That is, the sheath 120 contacts the entire wound surface and protectively covers the same and seals it from contamination and infection. Depending on the size and depth of the incision, the user can roll up the sheath 120 by gripping the outer ring 130 and turning it in a direction 135 until the sheath 120 abuts the outer edge of the wound opening 40. It is appreciated that the outer ring 130 can be turned around its annular axis in either an outward or inward direction to roll the sheath 120.

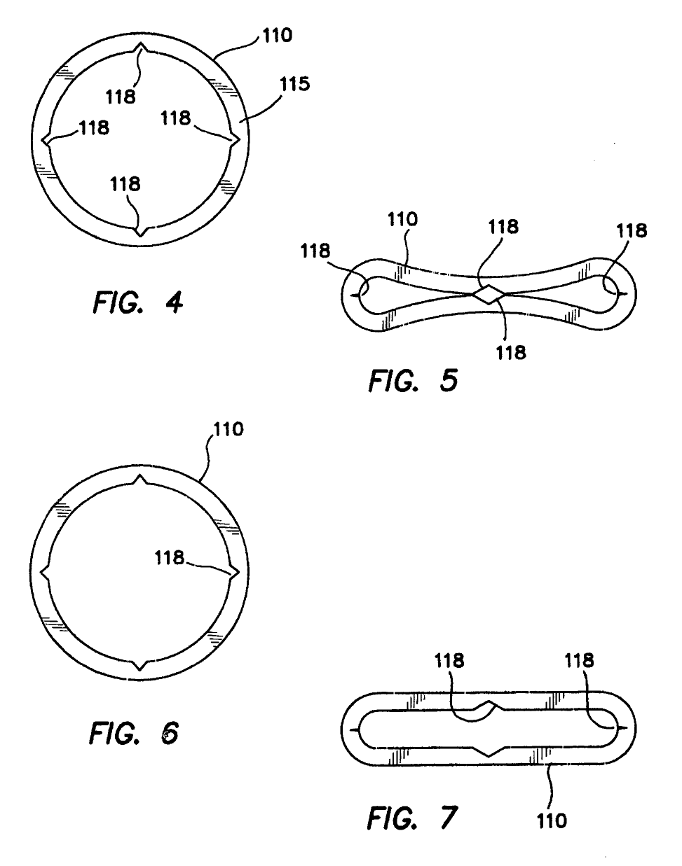

A feature of the invention is the inner flexible ring 110 may be folded, collapsed or shaped in a “low-profile” condition prior to insertion into the incision 20 so that it will correspond to the size and orientation of the incision 20. The inner ring 110 is then allowed to assume its original shape or condition once placed inside the body cavity 40. When removal is desired, the ring 110 may again be folded, collapsed or shaped to the “low-profile” condition when sheath-tension is released. Referring to FIGS. 4-7, there are shown top views of embodiments of the flexible inner ring 110 of the invention. The flexible inner ring 110 has an overall diameter and a cord 115 also having a diameter. The cord 115 may have a plurality of notches 118 or areas of removed material that facilitate folding or collapsing of the cord 115 at preferred locations. The flexible inner ring 110 may be squeezed between two points, for example at two opposing notches 118, causing the entire ring to elongate along a plane as illustrated in FIGS. 5 and 7. The ring may then be inserted or removed through a small surgical incision 20.

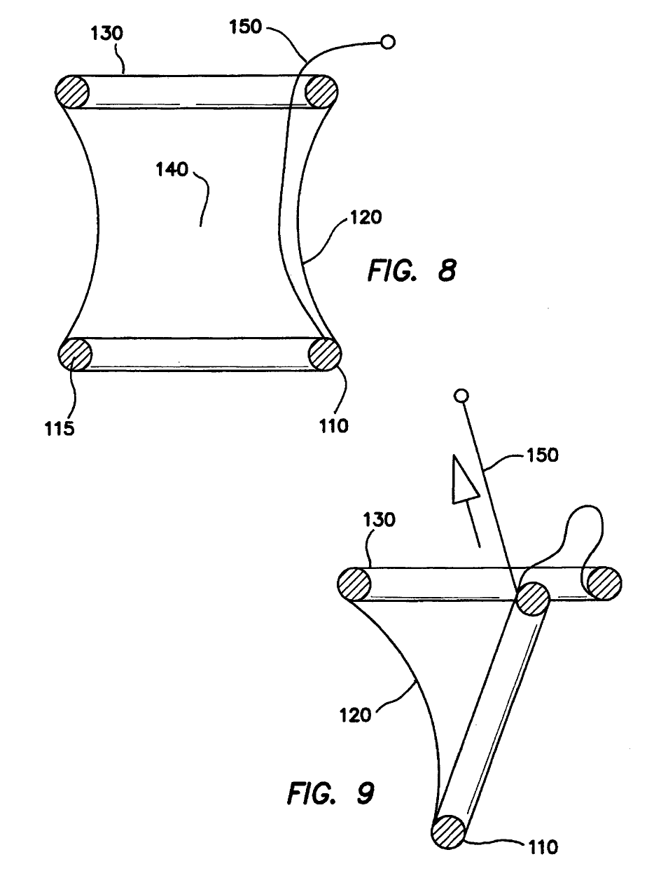

In another embodiment of the invention as illustrated in FIGS. 8 and 9, a tether 150 may be attached with the inner flexible ring 110 to further facilitate insertion and removal of the ring 110 from the incision 20. In particular, the tether 150 may have a length, a first end attached to the inner ring 110 interiorly of the sheath 120, and a second end disposed outside of the surgical incision 20, the tether 150 operating to facilitate removal of the inner ring 110 through the surgical incision 20. With this aspect, the first end of the tether 150 may be attached at one of the notches 118. It is appreciated that as the tether 150 is pulled through the surgical incision 20, the inner ring 110 deforms to a low-profile.

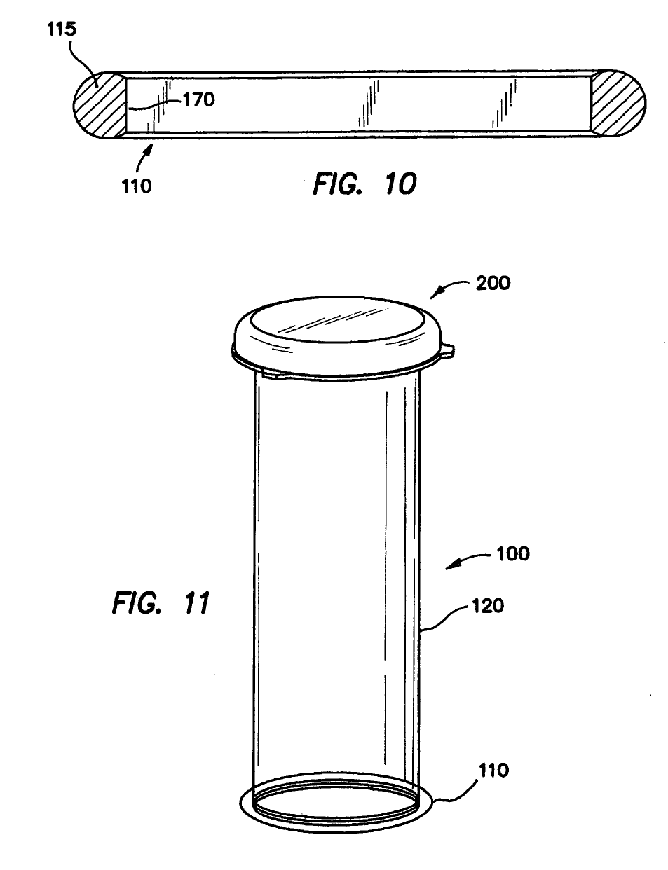

Referring now to FIG. 10, it should also be noted that the cord 115 of the flexible inner ring 110 may also have a cross-section that is not circular. In one embodiment, the cross section has a shape of a letter “D” that allows the flexible inner ring 110 to collapse to a lower profile than a circular profile.

Referring to FIG. 11, there is shown a perspective view of the wound retractor 100 adapted to be used with a hand assisted laparoscopic (HAL) device in accordance with another aspect the invention. With this aspect, access into and out of a patient's body is achieved by a HAL procedure using a surgical access device such as a Gelport® device 200 described in applicant's patent application Ser. No. 10/381,220, while retraction is provided by the wound retractor 100 of the present invention. Applicant's patent application Ser. No. 10/381,220 was filed on Mar. 20, 2003, claiming priority to international application PCT/US01/29682, filed on Sep. 21, 2001, both entitled “Surgical Access Apparatus and Method,” and both are incorporated herein by reference. This aspect of the invention combines features of both the wound retractor 100 and the surgical access device (Gelport®) 200 as described in the above mentioned patent application. As explained in the patent application, the current surgical access device uses a polyisoprene sheath that is wrapped distally around an O-ring, and once placed into a wound incision, the sheath is then stretched over extended tabs onto an abdominal base. The sheath of that surgical access device requires stretching and often times requires multiple attempts to secure it to the abdominal base. A novelty of this embodiment is to modify the cap and/or the abdominal base of the surgical access device 200 so that it will accept the wound retractor 100 of the present invention to replace the polyisoprene sheath and to maintain an airtight seal. That is, the use of the wound retractor 100 would simplify the HAL procedure and would not require stretching.

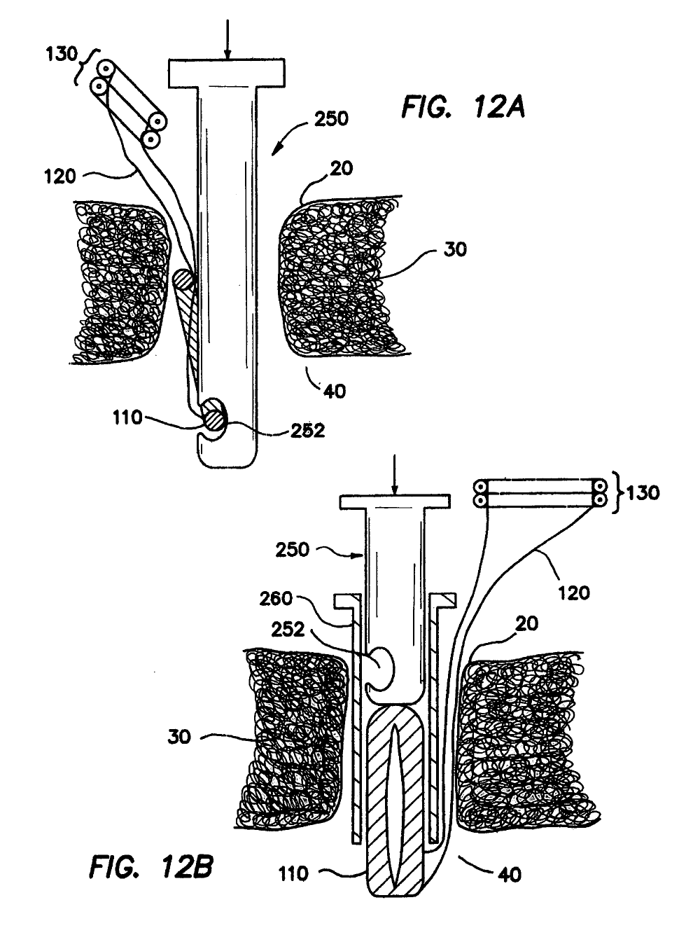

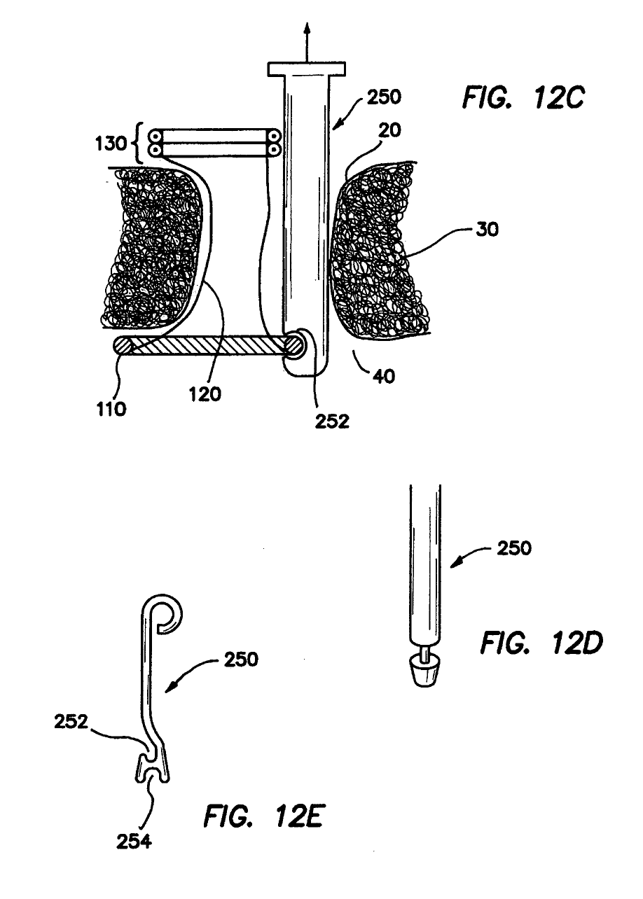

In yet another aspect of the invention, FIG. 12(a) illustrates a side view of an obturator 250 having a removal portion or hook 252 to facilitate placement of the inner flexible ring 110 of the wound retractor 100 through the incision 20 in the body wall 30 and into the body cavity 40. Specifically, the obturator 250 is used to introduce the inner flexible ring 110 through the incision 20 instead of using a surgeon's fingers or hand. FIG. 12(b) illustrates that placement of the inner flexible ring 110 can also be done through a cannula 260 using the obturator 250 of the invention. With this aspect, the inner flexible ring 110 is placed in the cannula 260 as shown in FIG. 12(b), which is then placed through the incision 20 in the body wall 30 and into the body cavity 40. The obturator 250 is then used to introduce the inner flexible ring 110 in the body cavity 40. A feature of this aspect is the inner flexible ring 110 can be folded, collapsed or shaped in a “low-profile” condition in the cannula 260 prior to insertion into the incision 20. It is appreciated that cannula 260 has a smaller profile than a typical surgeon's hand and, as such, a smaller incision is required to place the inner flexible ring 110 of the wound retractor 100.

After the surgical procedure is complete, the obturator 250 can be removed as illustrated in FIG. 12(c). In particular, the obturator 250 can be inserted into the incision 20 along either the exterior or interior side of the sheath 120 until the removal portion or hook 252 is adjacent to the inner ring 110, the removal portion or hook 252 can then be used to capture and remove the inner ring 110 from the body cavity 40 and through the body wall 30. The obturator 250 of FIGS. 12(a)-12(c) has a blunt tip but it is appreciated that it can also have an insertion portion or hook 254 as illustrated in FIG. 12(e). The obturator 250 is formed of a rigid material including a plastic or a metallic material. FIG. 12(d) illustrates another aspect of the obturator 250 having a recessed diameter to facilitate insertion and removal of the inner ring 110. In another aspect, the obturator 250 may further comprise a mechanical means including a sliding member that closes off at least one of the removal hook 252 and the insertion hook 254.

Many alterations and modifications may be made by those having ordinary skill in the art without departing from the spirit and scope of the invention. For these reasons, the above description should not be construed as limiting the invention, but should be interpreted as merely exemplary of preferred embodiments.

1. A surgical wound retractor adapted to dilate a surgical wound incision to a desired diameter, comprising:
a first ring having a diameter greater than the desired diameter of the wound incision and being adapted for disposition interiorly of the wound incision;
a second ring having an annular axis and a diameter greater than the desired diameter of the wound incision and being adapted for disposition exteriorly of the wound incision; and
a flexible sleeve disposed in a generally cylindrical form between the first ring and the second ring,
the first ring having at least one notch to facilitate folding or collapsing of the first ring during insertion and removal of the first ring through the wound incision.

2. The wound retractor of claim 1, wherein the first ring further comprises a second notch disposed on an opposing end of the first notch to further facilitate folding or collapsing of the first ring.

3. The wound retractor of claim 2, wherein the first ring is folded or collapsed by squeezing between the first and second notches.

4. The wound retractor of claim 1, further comprising a tether having a length, a first end attached to the first ring interiorly of the sheath, and a second end disposed outside of the wound incision, wherein the tether facilitates removal of the first ring through the wound incision.

5. The wound retractor of claim 4, wherein the first end of the tether is attached at the notch.

6. The wound retractor of claim 4, wherein the first ring deforms to a low-profile as the tether is pulled through the wound incision.

7. The wound retractor of claim 1, wherein the first ring has an annular cross-section that is circular, oval, D-shape, or any geometrical shape with multiple sides.

8. The wound retractor of claim 1, wherein the second ring may be rolled over itself and around the annular axis to provide the sleeve with a radial retraction force sufficient to stretch the incision to the desired diameter.

9. The wound retractor of claim 1, wherein the sleeve is made of a material that is flexible and impermeable to fluids and bacteria.

10. A surgical access device facilitating a sealing relationship with an instrument extending through the device and into an incision, the access device comprising:
a first ring being adapted for disposition interiorly of the incision;
a second ring having an annular axis and being adapted for disposition exteriorly of the incision;
a flexible sleeve connecting the first ring and the second ring; and
a valve structure disposed relative to the incision to securely receive the second ring,
the first ring having at least one notch to facilitate folding or collapsing of the first ring during insertion and removal of the first ring through the incision.

11. The surgical access device of claim 10, wherein the second ring has properties to roll over itself and around the annular axis to shorten the sleeve in predetermined increments.

12. The surgical access device of claim 11, wherein the rolling properties of the second ring provide the sleeve with a radial retraction force sufficient to stretch the incision to a desired diameter.

13. The surgical access device of claim 10, wherein the valve structure comprises a gel cap and an abdominal base.

14. The surgical access device of claim 13, wherein the gel cap further comprises a gel pad and a circumferential cap ring.

15. The surgical access device of claim 13, wherein the abdominal base comprises a rounded end surface along its inner diameter to secure the second ring.

16. The surgical access device of claim 10, wherein the first ring further comprises a second notch disposed on an opposing end of the first notch to further facilitate folding or collapsing of the first ring.

17. The surgical access device of claim 10, further comprising a tether having a length, a first end attached to the first ring interiorly of the sheath, and a second end disposed outside of the incision, wherein the tether facilitates removal of the first ring through the incision.

18. The surgical access device of claim 17, wherein the first end of the tether is attached at the notch.

19. The surgical access device of claim 17, wherein the first ring deforms to a low-profile as the tether is pulled through the incision.

20. The surgical access device of claim 10, wherein the first ring has an annular cross-section that is circular, oval, D-shape, or any geometrical shape with multiple sides.

21. A method for operating a surgical wound retractor, comprising:
providing a flexible sleeve having a first end attached to a first ring and a second end attached to a second ring, the first ring having at least one notch to facilitate folding or collapsing of the first ring;
folding or collapsing the first ring at the notch and inserting the first ring and the sleeve into a patient's body cavity so as to provide a channel from the outside to the inside of the patient's body;
drawing the second end of the sleeve upwards and radially outward or inward thereby drawing the first ring tightly against the inner surface of the body cavity, and thereby retracting the adjacent tissue and producing a tightly sealed opening in the body cavity during surgery; and
after surgery, grabbing the first ring so as to fold it at the notch and pulling the first ring and the sleeve through the wound opening.

22. The method of claim 21, the second ring having properties to roll over itself and around an annular axis to shorten the sleeve in predetermined increments.

23. The method of claim 22, further comprising the step of rolling the sleeve by gripping the second ring and turning it around the annular axis in either an outward or inward direction until the sleeve abuts the outer edge of the wound incision.

24. The method of claim 21, further comprising a tether having a length, a first end attached to the first ring interiorly of the sheath, and a second end disposed outside of the wound incision, wherein the tether facilitates removal of the first ring through the wound incision.

25. The method of claim 24; wherein the first end of the tether is attached at the notch.

26. The method of claim 24, further comprising the step pulling the tether at the second end to remove the first ring from the wound incision.

27. The method of claim 26, wherein the first ring deforms to a low-profile as the tether is pulled through the wound incision.

28. A surgical access device adapted to dilate a surgical wound incision to a desired diameter, comprising:
a wound retractor including:
a first ring having a diameter greater than the desired diameter of the wound incision and being adapted for disposition interiorly of the wound incision;
a second ring having an annular axis and a diameter greater than the desired diameter of the wound incision and being adapted for disposition exteriorly of the wound incision; and
a flexible sleeve disposed in a generally cylindrical form between the first ring and the second ring; and
an obturator having at least one of an insertion portion and a removal portion to facilitate insertion and removal of the first ring through the wound incision.

29. The access device of claim 30, further comprising a cannula to facilitate insertion of the first ring through the wound incision, wherein the first ring is folded or collapsed into the cannula prior to insertion through the wound incision.

30. The access device of claim 28, wherein the obturator is formed of a rigid material including a plastic or metallic material.

31. The access device of claim 28, wherein the obturator further comprises a mechanical means including a sliding member that closes off at least one of the insertion portion and the removal portion.
